(19) United States
(12) Patent Application Publication
(10) Pub. No.: US 2017/0312329 A1
(43) Pub. Date: Nov. 2, 2017

(54) Compositions and Methods for Treatment of Prediabetes

(71) Applicant: William H. Cross, III, Waco, GA (US)

(72) Inventor: William H. Cross, III, Waco, GA (US)

(21) Appl. No.: 15/650,825

(22) Filed: Jul. 14, 2017

Related U.S. Application Data
(63) Continuation-in-part of application No. 15/496,919, filed on Apr. 25, 2017.
(60) Provisional application No. 62/327,421, filed on Apr. 25, 2016.

(51) Int. Cl. A61K 36/87; A61K 31/593; A61K 31/4172; A61K 31/221; A61K 31/047; A61K 31/198; A61K 31/155; A61K 31/145; A61K 31/714; A61K 31/385

(52) U.S. Cl. CPC A61K 36/87; A61K 31/714; A61K 31/593; A61K 31/4172; A61K 31/385; A61K 31/047; A61K 31/198; A61K 31/155; A61K 31/145; A61K 31/221

(57) ABSTRACT

The invention provides compositions and methods to treat prediabetes. In particular, the inventor has discovered that particular combinations of diverse types of antioxidants have synergistic and surprising effects for use against prediabetes. The composition includes: a plurality of heteroatom-based antioxidants having the formula X—CH₂—CH(R)—(CH₂)ₘ—Z; a plurality of antioxidants with a conjugated segment; an antioxidant that is a flavan-3-ol; and an antioxidant that contains a disulfide bond. The antioxidants are chosen such that at least one has a pro-oxidative effect.

### RELATED APPLICATIONS

This application is a continuation in part of nonprovisional U.S. patent application Ser. No. 15/496,919, filed Apr. 25, 2017, which derives priority from provisional U.S. patent application Ser. No. 62/327,421, filed Apr. 25, 2016, both having the same title and inventor as the present application.

### FIELD OF THE INVENTION

The invention concerns compositions and methods for the treatment and prevention of prediabetes.

### BACKGROUND

Prediabetes—formerly known as borderline diabetes—is a metabolic condition affecting 86 million Americans and characterized by blood sugar levels that are higher than the normal range yet below those of diabetes mellitus. Prediabetic patients lack one or more symptoms that characterize diabetes, but the condition is often a prelude to type 2 diabetes mellitus (T2DM). A quarter of the cases commonly progress to T2DM within 3 to 5 years, and half within 10 years. Prediabetes also often has a role in heart disease and stroke. Prediabetes sometimes may be identical to metabolic syndrome, but the latter is evaluated by a different set of biomarkers.

A common symptom of prediabetes is impaired fasting glucose (IFG), in which blood sugar levels are high but still below those typical of diabetes. In IFG the fasting blood glucose or the 3-month average blood glucose (A1C) is elevated above normal levels but not extreme. It is associated with insulin resistance and an increased risk of heart disease and mortality.

Some IFG patients respond normally to a glucose tolerance test, whereas others have impaired glucose tolerance (IGT). IGT may precede T2DM by many years, and like IFG it is associated with insulin resistance. IGT is a more reliable indicator than IFG for increased risk of heart disease and mortality. Among American adults over 40 years of age, 33% have IFG, 15% have IGT, and 40% are prediabetic (i.e., they have IFG and/or IGT). Children have similar risks.

Not all cases of prediabetes can be diagnosed by IFG or IGT alone, so a range of symptoms are monitored: persistent hunger; obesity, especially abdominal or visceral obesity, and especially a body mass index over 25; weight gain or at the other extreme, unexplained weight loss; flu-like weakness and fatigue; blurry vision; slowness of healing for cuts and bruises; tingling or insensate extremities; frequency of urination; and recurring infections of the skin, gums, bladder or vagina.

The diagnostic tests include the fasting plasma glucose (FPG) test: prediabetic blood sugar is in the range of 100-125 mg/dL, equivalent to 5.6 mM/L to 6.9 mM/L (numbers above those indicate diabetes). The oral glucose tolerance test (OGTT) follows immediately after the fasting test just described and administers 75 grams of a sugary solution. Two hours later the blood sugar is tested again: 140-199 mg/dL indicates prediabetes (or diabetes if the number is higher). The hemoglobin A1C test, also known as the average blood sugar test, HbA1c test or glycohemoglobin test, determines average percentage of hemoglobin in the blood that is attached to glucose over the recent two to three months: 5.7% to 6.4% is diagnostic for prediabetes; above that indicates diabetes (or uncontrolled diabetes if it is being treated).

Prediabetes is associated with many conditions, including sleep disorders, genetics, cardiovascular disease, hypertension, high triglyceride levels, low HDL cholesterol, elevated weight, pregnancy and gestational diabetes, high birth weight, polycystic ovarian syndrome, and race (especially for African-Americans, Hispanic Americans, Native Americans, and Asian Americans). Among the lifestyle intervention guidelines to prevent the onset of T2DM are: dietary regimes that are low in sugar, refined carbohydrates, saturated fats, salt and total calories; regular physical exercise, for instance 30 minutes per day five days per week; and weight reduction even by just a few percent. Such measures can also cure prediabetes in at least some cases, though more exercise is recommended, such as 45 minutes per day.

Early intervention is urgent, both because diagnosis often misses prediabetes, and because the type of damage caused by T2DM may already be starting even in the prediabetic stage. Diabetic types of effects include atherosclerosis within the cardiovascular larger arteries. Other effects occur at the small blood vessels, with particular damage noted in the retinas, the kidneys, and the nerves. Currently metformin is the only drug recommended by the American Diabetes Association to prevent the onset of T2DM in prediabetic patients.

The incidence of prediabetes remains high despite widespread monitoring and health education. And methods to treat it have been lacking apart from the lifestyle changes just discussed. Thus there is an ongoing need for compositions to treat and prevent prediabetes.

### BRIEF SUMMARY OF THE INVENTION

The invention provides compositions and methods to treat and prevent development of prediabetes. In particular, the present invention has discovered that combining certain diverse types of antioxidants yields synergistic and surprising effects against prediabetes. These antioxidant types include: heteroatom-based antioxidants from a particular chemical structural antioxidants with a conjugated segment of alternating single and double bonds; antioxidants with a disulfide bond; and antioxidants having a flavan-3-ol skeleton. It has further been discovered that it is beneficial to include one or more antioxidants that facilitates an oxidative balance, i.e., that is also a pro-oxidant.

In a particular embodiment the invention provides an improved composition for treatment of prediabetes comprising therapeutically effective amounts of at least four types of antioxidant compounds, characterized in that:

a) a plurality of heteroatom-containing natural antioxidant compounds have the formula

X—CH(R¹)—CH(R²)—(CH₂)ₘ—Z,

wherein:
i) X is one of the following moieties or a pharmaceutically acceptable salt thereof: —NH₂ or —N(CH₃)₃;
ii) R¹ is —H, —CO₂H, or a pharmaceutically acceptable salt of —CO₂H;
iii) R² is —H, —OH, or —O—acetyl;
iv) m is 0, 1, 2, or 3; and
v) Z is one of the following moieties or a pharmaceutically acceptable salt thereof: —SH, —SO₂H, —SO₃H, —CO₂H, —N═C(—NH₂)—NH₂, —CH(—NH₂)—CO₂H; —imidazolyl; or —C(═O)—NH—CH(—CO₂H)—CH₂—Y where Y is 4-imidazolyl or 1-methyl-4-imidazolyl;

b) a plurality of antioxidant compounds have a conjugated segment, comprising at least one vitamin D compound and at least one cobalamin compound;

c) at least one of the antioxidant compounds comprises a disulfide bond; and

d) at least one of the antioxidant compounds is a flavan-3-ol compound;

wherein at least one antioxidant compound in the composition is also a pro-oxidant.

In a further embodiment the invention provides a method for treatment of prediabetes comprising administration of a composition that comprises therapeutically effective amounts of at least four types of antioxidant compounds, characterized in that:

a) a plurality of heteroatom-containing natural antioxidant compounds have the formula

X—CH(R¹)—CH(R²)—(CH₂)ₘ—Z,

wherein:
i) X is one of the following moieties or a pharmaceutically acceptable salt thereof: —NH₂ or —N(CH₃)₃;
ii) R¹ is —H, —CO₂H, or a pharmaceutically acceptable salt of —CO₂H;
iii) R² is —H, —OH, or —O—acetyl;
iv) m is 0, 1, 2, or 3; and
v) Z is one of the following moieties or a pharmaceutically acceptable salt thereof: —SH, —SO₂H, —SO₃H, —CO₂H, —N═C(—NH₂)—NH₂, —CH(—NH₂)—CO₂H; —imidazolyl; or —C(═O)—NH—CH(—CO₂H)—CH₂—Y where Y is 4-imidazolyl or 1-methyl-4-imidazolyl;

b) a plurality of antioxidant compounds have a conjugated segment, comprising at least one vitamin D compound and at least one cobalamin compound;

c) at least one of the antioxidant compounds comprises a disulfide bond; and

d) at least one of the antioxidant compounds is a flavan-3-ol compound;

wherein at least one antioxidant compound in the composition is also a pro-oxidant.

### DETAILED DESCRIPTION OF THE INVENTION

The invention may be better understood by consideration of the following definitions of the terms as used herein.

The term “composition” as used with reference to compositions of the invention means a food supplement composition or pharmaceutical composition.

The term “prediabetes” means a metabolic condition identified by at least one of the following: fasting plasma glucose level in the range of 100-125 mg/dL; blood sugar in an oral glucose tolerance test in the range of 140-199 mg/dL; or hemoglobin A1C in the range of 5.7% to 6.4% in a hemoglobin A1C test.

The terms “diabetes” means a metabolic condition identified by at least one of the following: fasting plasma glucose level ≧126 mg/dL; blood sugar in an oral glucose tolerance test ≧200 mg/dL; or hemoglobin A1C≧6.5% in a hemoglobin A1C test.

The term “symptom of prediabetes” means a symptom that is commonly used by the allopathic medical community to monitor or diagnose prediabetes. Examples of symptoms of prediabetes include: IFG; IGT; persistent hunger; obesity; abdominal obesity; visceral obesity; a body mass index over 25; weight gain; unexplained weight loss; flu-like weakness; fatigue; blurry vision; slowness of healing for cuts; slowness of healing for bruises; a tingling extremity; an insensate extremity; frequent need to urinate; a recurring infection of the skin; a recurring infection of the gums; a recurring infection of the bladder; a recurring infection of the vagina; blood sugar in the range of 100-125 mg/dL or 5.6 mM/L to 6.9 mM/L for the fasting plasma glucose test; blood sugar in the range of 140-199 mg/dL for the oral glucose tolerance test; or average hemoglobin A1C of 5.7% to 6.4% in the hemoglobin A1C test.

The term “reduce or mitigate” as used with respect to a symptom of prediabetes means to decrease the manifestation of the symptom by an amount that is customarily considered medically significant, measurable and relevant. In a particular embodiment the reduction or mitigation decreases the deviation from a healthy normal range by at least 10%.

The terms “method” as used with respect to treatment of prediabetes or its symptoms according to the invention means a protocol to reduce or mitigate the symptoms thereof by providing a composition of the invention. The term “treatment” as used with respect to prediabetes means use of such a method.

The term “administration of a composition” means to provide a dose of the composition for ingestion by someone in need thereof.

The term “antioxidant” as used with respect to a compound means that the molecule can inhibit oxidation or reactions promoted by oxygen, peroxides, or free radicals. Important examples of antioxidant compounds include vitamin C, vitamin E, phenols, and zinc. Various types of antioxidant activity exist. Some antioxidants such as zinc donate an electron to reduce a free radical or oxidizing species. Some antioxidants bond to a free radical species to terminate a radical chain reaction. Some antioxidants such as phenols donate a hydrogen atom to a free radical species to terminate a radical chain reaction and form a stabilized radical that then reacts by another type of reaction to eliminate the radical.

The term “pro-oxidant” as used with respect to a compound means that the molecule can promote oxidation or other reactions that are commonly mediated by oxygen, peroxides, or free radicals. This property is counterintuitive for antioxidants but several examples are known, particularly for vitamins C and E. Thus for instance ascorbate (vitamin C) reduces Fe(III) to produce Fe(II), which can then react with hydrogen peroxide (an oxidizing substance) by the Fenton reaction to generate hydroxyl free radicals by the following redox cycling reactions: 2 Fe²⁺+2H₂O₂→2Fe³⁺+2OH.+2OH⁻; 2Fe³⁺+Ascorbate→2Fe²⁺+Dehydroascorbate. Thus antioxidants that can serve as reduction agents may have a pro-oxidant effect at suitable concentrations and if O₂ or peroxide and a transition metal are present.

The terms “pro-oxidative effect” and “pro-oxidant activity” as used with respect to a compound's effect are used synonymously and mean that the molecule acts as a pro-oxidant in that environment. Pro-oxidative effects may be associated with the generation of oxidative stress in tissues, however in some cases that stress may mimic a normal physiological signal, for instance for the conversion of fibroblasts to myofibroblasts in fibrosis or scar formation. Where an antioxidant has a pro-oxidative effect, the pro-oxidative effect may be different in mechanism and strength from that of the antioxidant activity of the molecule.

The term “pro-oxidative effect in the liver” as used with respect to a compound means that the molecule has pro-oxidant activity that is known to occur at least in the liver. In particularly useful embodiments of the invention one or more of the antioxidants that comprise stabilizing heteroatoms have pro-oxidant activity in the liver.

The term “type” as used with respect to an antioxidant compound means its structural category. As defined herein, types of antioxidant compound include at least: heteroatom-containing natural antioxidant compounds have the formula X—CH(R¹)—CH(R²)—(CH₂)ₘ—Z; those comprising a conjugated segment; those comprising a disulfide bond; and those having a flavan-3-ol skeleton. Typically there is no overlap between these categories, however the invention is not so limited.

The term “heteroatom-containing natural antioxidant compound” means a natural compound that is an antioxidant and that contains a heteroatom. As used herein this description is coupled with a generic chemical formula X—CH(R¹)—CH(R²)—(CH₂)ₘ—Z, wherein:
i) X is one of the following moieties or a pharmaceutically acceptable salt thereof: —NH₂ or —N(CH₃)₃;
ii) R¹ is —H, —CO₂H, or a pharmaceutically acceptable salt of —CO₂H;
iii) R² is —H, —OH, or —O—acetyl;
iv) m is 0, 1, 2, or 3; and
v) Z is one of the following moieties or a pharmaceutically acceptable salt thereof: —SH, —SO₂H, —SO₃H, —CO₂H, —N═C(—NH₂)—NH₂, —CH(—NH₂)—CO₂H; —imidazolyl; or —C(═O)—NH—CH(—CO₂H)—CH₂—Y where Y is 4-imidazolyl or 1-methyl-4-imidazolyl.
   The chemical notations, terminology, and structural designations there follow the conventions of the art of chemistry, except that the identities X, Y, and Z are understood to correspond to moieties that are specific to the individual compound but vary across the category.

The term “heteroatom” means any atom in an organic molecule that is not carbon or hydrogen, where the term “organic” as used with respect to a molecule has its usual and ordinary meaning in organic chemistry. Illustrative examples of heteroatoms include but are not limited to nitrogen, oxygen, phosphorus, sulfur, selenium, boron, fluorine, chlorine, bromine, and iodine, as those atoms are named on the periodic table.

The term “stabilizing heteroatom” as used with respect to an antioxidant compound means a heteroatom that is capable of donating electron density toward a radical on a neighboring carbon atom in a molecule, and or that possesses an empty orbital to accept electron density from such a radical. Examples of stabilizing heteroatoms include boron, nitrogen, oxygen, phosphorus and sulfur but the invention is not so limited. Preferred stabilizing heteroatoms are nitrogen, oxygen and sulfur, and a particularly preferred stabilizing heteroatom is sulfur but the invention is not so limited.

The term “saturated carbon” as used with respect to an atom in a compound means that it is a carbon atom for which bonds to its neighboring atoms are all and only single bonds, wherein the recited carbon atom is bonded to at least one other carbon atom and to at least one hydrogen atom. As used herein, the term “saturated carbon” contemplates that the carbon atom may be covalently bonded to one or more heteroatoms, but does not necessarily have such a bond.

The term “adjacent to” as used with respect to one atom and another in a compound means that the two are covalently bonded to one another.

The terms “taurine”, “beta-alanine”, “acetyl-carnitine”, “agmatine”, “carnosine” “cysteine”, “sulfinoalanine”, “hypotaurine”, “carnitine”, “lysine”, “anserine”, “histidine”, “arginine”, and “ornithine” have their usual and ordinary meanings in the fields of chemistry and biochemistry.

The term “conjugated segment” as used with respect to a molecule means a portion of the molecular skeleton that has single bonds alternating with multiple bonds such as double or triple bonds. In particular embodiments the skeleton of the conjugated segment is composed entirely of carbon atoms. In further embodiments the skeleton of the conjugated segment is composed of carbon atoms and one or more heteroatoms. In preferred embodiments the conjugated segment is covalently bonded to at least one saturated carbon atom such as a methyl group (i.e. —CH₃), a substituted methyl group (i.e., —CH₂X or —CHXY where X and Y are, e.g., halogen atoms that may be the same or different), a methylene group (i.e., —CH₂—), a singly substituted methylene group (i.e., —CHX—, where X is, e.g., a halogen atom), or a tertiary carbon that is bonded to hydrogen. Non-limiting illustrative examples of conjugated segments in compounds that are suitable for use with the invention include: the porphyrins rings of cobalamin compounds; the conjugated portions of rings, branches and linear backbone in vitamin D compounds such as ergocalciferol (vitamin D₂) and cholecalciferol (vitamin D₃); and the linear chains of the carotenoids, as well as cyclic end groups of the carotenoids to the extent that they possess a C═C double bond conjugated with the linear chain.

The term “vitamin D compound” means a molecule that is within the group known as vitamin D. These are secosteroids and in particular include the following: 7-dehydrocholesterol, also known as previtamin-D₃; vitamin D₂, also known as ergocholecalciferol; vitamin D₃, also known as cholecalciferol and activated 7-dehydrocholesterol; 25-D, also known as 25-hydroxyvitamin D, calcidiol, and calcifediol; and 1,25-D, also known as 1,25-dihydroxycholecalciferol, 1,25-dihydroxyvitamin D₃, and calcitriol.

The term “cobalamin compound” means a compounds having a skeleton of cobalamin (i.e., vitamin B₁₂, i.e., α-(5,6-dimethylbenzimidazolyl)cobalamide) with an axial ligand on the cobalt atom, where the ligand is not bridged to the rest of the molecule. In nonlimiting illustrative examples that axial ligand is: —CH₃ for methylcobalamin; —OH for hydroxocobalamin; —CN for cyanocobalamin; and -Ado for adenosylcobalamin, where -Ado is an adenosyl moiety. In a particular embodiment of the invention methylcobalamin is the form of cobalamin used, but the invention is not so limited. As used herein, the term “vitamin B₁₂” is used synonymously with cobalamin, and includes all active forms of cobalamin. When a weight quantity of cobalamin or vitamin B₁₂ is given herein, it will be understood to refer to the weight quantity of the particular cobalamin species under consideration, including the R-group, regardless of which R-group is appended.

The terms “vitamin D₃” and “methylcobalamin” have their usual and ordinary meanings in the fields of chemistry and biochemistry.

The term “carotenoid” has its usual and ordinary meaning in natural products chemistry.

The terms “carotene”, “lycopene”, “neurosporene”, “phytofluene”, “phytoene”, “xanthophyll”, “canthaxanthin”, “cryptoxanthin”, “zeaxanthin”, “astaxanthin”, “lutein”, and “rubixanthin” have their usual and ordinary meaning in the fields of chemistry and natural products.

The term “lettered carotenes” means carotene compounds designated by Greek letters and for example includes alpha-carotene, beta-carotene, gamma-carotene, delta-carotene, epsilon-carotene and zeta-carotene.

The term “disulfide bond” as used with respect to a molecule means a single covalent bond that links two neighboring divalent sulfur atoms, forming an —S—S— moiety in a molecule. As used herein with respect to a sulfur atom, the phrase “is not part of a disulfide bond” means that the atom is not part of an —S—S— moiety, whereas a sulfur atom that is part of a disulfide bond is part of an —S—S— moiety.

The terms “alpha-lipoic acid”, “lipoamide”, “glutathione disulfide”, “cystine”, “asparagusic acid”, “lenthionine”, “ajoene”, “allithiamine”, “allyl propyl disulfide”, “diallyl disulfide”, “fursultiamine”, “pantethine”, “prosultiamine”, “pyritinol”, and “sulbutiamine” have their usual and ordinary meanings in the fields of chemistry and biochemistry.

The term “phenolic group” as used with respect to a molecule means a benzene ring that bears at least one hydroxy (—OH) substituent. In certain embodiments the benzene ring may be a fused ring, for instance such as is found in flavan-3-ols. In certain embodiment the phenolic group may be part of a condensed phenolic system such as tannic acid or ellagic acid. Non-limiting illustrative examples of phenolic substituents that may be found in ingredients according to the invention include those bonded to the ring or by an ether or ester of phenol (hydroxybenzene), pyrocatechol (o-dihydroxybenzene), resorcinol (m-dihydroxybenzene), pyrogallol (1,2,3 -trihydroxybenzene), or phloroglucinol (1,3,5 -trihydroxybenzene). In some embodiments the phenol has a C—C or C—O bond to a sugar moiety.

The term “flavan-3-ol compound” means a natural derivative of flavan-3-ol, i.e., it has moieties bonded to the 2-phenyl-3,4-dihydro-2H-chromen-3-ol skeleton shown below.

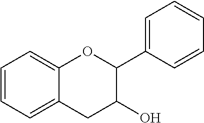 [SMILES: OC1Cc2ccccc2OC1c1ccccc1]

The moieties are typically hydroxyl groups on the aromatic rings, but may include covalent bonds that form an oligomer or polymer, and in some cases may include sugar groups as in a glycoside. The term flavan-3-ol compound as used herein includes but is not limited to catechin (C), epicatechin (EC), gallate (G), epigallocatechin (EGC), epigallocatechin gallate (EGCG), theaflavins, theaflavin-3-gallate, thearubigins, and stereoisomers and glycosides of those compounds. The term flavan-3-ol compound as used herein further includes oligomers and polymers of those compounds, in particular the proanthocyanidins and procyanidins. The terms “tannin”, “condensed tannin”, “hydrolysable tannin”, “proanthocyanidin”, “procyanidin”, “prodelphinidin”, “proguibourtinidin”, and “prorobinetinidin” as used herein have their usual and ordinary meanings in natural products and plant biochemistry, and are for instance consistent with the respective usage of such terms in CHEMISTRY AND SIGNIFICANCE OF CONDENSED TANNINS, Richard W. Hemingway and Joseph J. Karchesy, eds. (1989).

The term “glycoside” as used with respect to a compound or moiety means that it has a sugar residue covalently bonded thereto; the sugar may be a monosaccharide, disaccharide, trisaccharide, or higher order saccharide.

The terms “catechin”, “epicatechin”, “epigallocatechin”, “epicatechin gallate”, “epigallocatechin gallate”, “epiafzelechin”, “fisetinidol”, “guibourtinidol”, “mesquitol”, and robinetinidol” refer to flavan-3-ol compounds and have their usual and ordinary meaning in the fields of organic chemistry and natural products, and include the various stereoisomers thereof. The generic chemical structure for catechins and epicatechins is shown below. Where the C-2 and C-3 carbons define a trans configuration the molecule is a catechin. Where they define a cis configuration the molecule is an epicatechin.

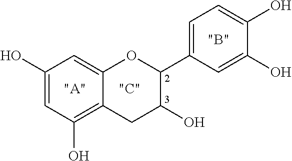 [SMILES: Oc1cc(O)c2c(c1)OC(c1ccc(O)c(O)c1)C(O)C2]

The term “derivative” as used with respect to a molecule means a variant in which the molecule is bonded to another chemical moiety.

The terms “monomer”, “oligomer”, and “polymer” as used herein with respect to a structural unit have their usual and ordinary meaning in the arts of chemistry, biochemistry and polymer science, where a monomer has one unit, an oligomer has at least two such units covalently bonded to one another, and a polymer has many such units. As an example, typically procyanidins are concatenated oligomers of catechins and or epicatechins, having from 2 to 50 or more structural units in a linear molecular chain. As a further example, proanthocyanidins, of which procyanidins are one subset, may further include gallate units, and their oligomers and polymers may be either linear or branched or in some cases cross-linked.

The term “extract” as used with respect to a botanical substance means a product that has been obtained by removal from a plant material but remains in a mixed form. Illustrative methods to obtain extracts include expression of oil; absorption by steeping the plant material in a solvent such as, e.g., water or ethanol; maceration of the plant material; and distillation. In some cases two or more of these methods are combined, e.g., maceration and distillation. Other methods include, for instance, spagyric extraction, often involving fermentation, distillation and extraction of mineral components from the ash of a plant. In particularly useful embodiments according to the invention the extract is a composite that includes flavan-3-ols extracted from plant tissue by means of a solvent such as water or ethanol. The plant tissue that has been extracted may be from the roots, leaves, stems, flowers, fruits, seeds, components of any of those, or another part of a plant.

The term “grape seed extract” means an extract of grape seed(s). In particularly useful embodiments of the invention the grape seed extract is a commercially available product, such as is sold by, e.g., the company Naturex, SA.

The term “therapeutically effective amount” as used with respect to an ingredient or composition means an amount sufficient to elicit a desired biological response. The therapeutically effective amount or dose will depend on the age, sex and weight of the patient, and the current medical condition of the patient. The skilled artisan will be able to determine appropriate dosages depending on these and other factors in addition to the present disclosure. By definition, an amount of a vitamin or mineral adequate to correct or maintain basal levels of the vitamin or mineral above a depressed or deficient state are therapeutically effective. Non-limiting examples of therapeutically effective amounts for the present invention include the 2013 dietary reference intakes based on dietary equivalents (“Des”) for vitamin D compounds, cobalamin compounds (2.4 μg/day), and carotenoids such as vitamin A. For purposes of the present invention therapeutically effective amounts may include any larger amount up to the maximum level of daily nutrient intake that is likely to pose no risk of adverse effects. The dietary reference intakes (DRI) for those, also known as recommended dietary allowances (“RDA”), are published by Food and Nutrition Board of the Institute of Medicine, National Academy of Sciences.

The term “pharmaceutically acceptable” as used with respect to salts and complexes of compounds herein, means those which are useful in preparing a pharmaceutical composition that is generally safe, non-toxic and neither biologically nor otherwise undesirable and includes both those which are acceptable for veterinary use as well as those which are acceptable for human pharmaceutical use. Pharmaceutically acceptable salts are for example acid addition salts and basic salts but also include inner salts such as zwitterions. Acid addition salts are, e.g., salts made by addition of an acid, such as by HCl, HBr, H₂SO₄, H₃PO₄ or another inorganic acid or by citric acid or another organic acid. Basic salts are, e.g., salts having a cation selected from alkali or alkaline metals, e.g., Na⁺, K⁺, or Ca²⁺, or an ammonium ion N³⁰ (R1)(R2)(R3)(R4), wherein R1 to R4 independently of each other mean: hydrogen, an optionally substituted C1-C6 alkyl group, an optionally substituted C2-C6 alkenyl group, an optionally substituted C6-C10 aryl group, or an optionally substituted C6-C10 heteroaryl group. Further examples of pharmaceutically acceptable salts are described in REMINGTON'S PHARMACEUTICAL SCIENCES, 18 ed. (1990) and in ENCYCLOPEDIA OF PHARMACEUTICAL TECHNOLOGY.

The terms and abbreviations “milligram” and “mg” are equivalent. The terms and abbreviations “microgram” and “mcg” and “μg” are equivalent. And terms of measurement conform to their respective meanings in the International System of Units, also known as the Système international d'unités.

The abbreviation “I.U.” as used with respect to amounts of an ingredient means International Unit(s) as they are stated in the art of pharmacology for biologically active quantities of vitamins, hormones, some medications, vaccines, blood products, and similar biologically active substances.

The inventor has discovered that combining antioxidants from four particular classes provides surprisingly good benefits against prediabetes and its symptoms. Although vitamin C and vitamin E are commonly provided in prior art antioxidant supplements, the present invention does not require them nor does either of those vitamins fall into one of the four classes herein. The discovery further finds that providing at least one antioxidant that can also serve as a pro-oxidant is beneficial. Without being bound by the mechanism it is theorized that prediabetes is associated with increased damage to biochemical intermediates and tissues from specific types and ratios of free radicals, and that the present invention addresses their relative reactivity and prevalence. Moreover the varieties of compounds taught for use here will distribute to different tissues and cellular components, and thus without being bound by theory are believed to address the damage in each of those places that represent a signature pattern of prediabetes and diabetes.

### CATEGORY I

### Heteroatom-based Natural Antioxidant Compounds

These fall within a generic structural family having a short (2-5 carbons) alkyl chain with at one end a simple nitrogen group such as an unsubstituted amine or tri-methyl ammonium group, and at the neighboring carbon optionally an —O-acetyl or carboxyl group, and at the other end a group from an acid or acid derivative, such as a sulfonic, carboxylic or amide group, optionally augmented by an acid and or diimidazole group. The inventor theorizes without being so limited that radical oxidizing species in the body abstract a H• atom from the alpha-carbon for the terminal amine of the antioxidant, and that the nitrogen atom is able to donate electron density (or in the case of the ammonium species, the nitrogen atom is able to donate electron density after a demethylation) to stabilize the adjacent radical. The radical may be further stabilized by donation from an acetyl ester oxygen at the same site. Or a captodative (“push-pull”) effect may stabilize the radical where an amine donates electron density from one side of the unpaired electron and a carboxyl center draws electron density from the other. Some molecules may have additional sited for stabilized abstraction of a hydrogen atom. This hypothesis assumes that the stabilized radical is less aggressively reactive and thus can be rehydrogenated or metabolized without perpetuating the more destructive branches of a reactive radical manifold. Again, the invention is not limited by this hypothetical mechanism.

In general it appears that it is most helpful to include a plurality of antioxidant compounds from this category, and the invention finds that it is useful to include as many as five or more. Preferred compounds include taurine, beta-alanine, acetyl-carnitine, agmatine, carnosine, cysteine, sulfinoalanine, hypotaurine, carnitine, lysine, anserine, histidine, arginine and ornithine. Particularly preferred compounds are taurine, carnosine, carnitine, acetylcarnitine, and agmatine, depicted in Table I below. However the invention is not so limited. Natural compounds, and particularly compounds having the stereoisomer found in the body are preferred, because they are safer in some cases (e.g., L-carnitine is less toxic than D-carnitine) and have an inherent advantage for docking with and being transported by or through natural stereoisomers of lipids, proteins, carbohydrates and nucleic acids. However the invention is not so limited.

In particular embodiments the composition is provided in a dose form. In certain embodiments the dose form includes taurine provided in a range selected from: 40 to 360 mg; 60 to 340 mg; 80 to 320 mg; 100 to 300 mg; 120 to 280 mg; 140 to 260 mg; 160 to 240 mg; 180 to 220 mg; or about 200 mg. In some embodiments the dose form includes beta-alanine provided in a range selected from: 10 to 90 mg; 15 to 85 mg; 20 to 80 mg; 25 to 75 mg; 30 to 70 mg; 35 to 65 mg; 40 to 60 mg; 45 to 55 mg; or about 50 mg. In various embodiments the dose form includes acetyl-L-carnitine provided in a range selected from: 20 to 180 mg; 30 to 170 mg; 40 to 160 mg; 50 to 150 mg; 60 to 140 mg; 70 to 130 mg; 80 to 120 mg; 90 to 110 mg; or about 100 mg. In particular embodiments the dose form includes agmatine provided in a range selected from: 25 to 225 mg; 40 to 210 mg; 55 to 195 mg; 70 to 180 mg; 85 to 165 mg; 100 to 150 mg; 115 to 135 mg; or about 125 mg. In further embodiments the dose form provides L-carnosine provided in a range selected from: 5 to 45 mg; 7.5 to 42.5 mg; 10 to 40 mg; 12.5 to 37.5 mg; 15 to 35 mg; 17.5 to 32.5 mg; 20 to 30 mg; 22.5 to 27.5 mg; or about 25 mg. In some embodiments the dose form provides an 8:2:4:5:1 respective ratio of taurine, beta-alanine, acetyl-L-carnitine, agmatine, and L-carnosine.

TABLE I
ANTIOXIDANTS WITH A STABILIZING HETEROATOM
Taurine

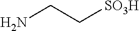

Beta-alanine

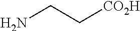

Agmatine

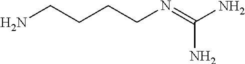

Acetyl-L-carnitine

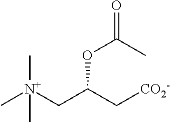

L-carnosine

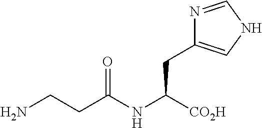 [SMILES: NCCC(=O)N[C@@H](Cc1c[nH]cn1)C(=O)O]

### CATEGORY II

### Natural Compounds Having a Conjugated Segment

Conjugation is the alternation of double and single bonds, and particularly where several double bonds are involved in the sequence, this provides a stabilized site for hydrogen abstraction at saturated carbon atoms that are adjacent to a double bond in the series, whether at the terminus of a conjugated segment, or at a branch along the chain. This is true even when the branch is as short as a methyl group.

The inventor has found that vitamin D compounds and cobalamin compounds are useful of this purpose, and that including a representative antioxidant compound from each of those categories is particularly useful. In certain embodiments at least one carotenoid is also included.

Useful vitamin D compounds for purposes of the invention include: 7-dehydrocholesterol, also known as previtamin-D₃; vitamin D₂, also known as ergocholecalciferol; vitamin D₃, also known as cholecalciferol and activated 7-dehydrocholesterol; 25-D, also known as 25-hydroxyvitamin D, calcidiol and calcifediol; and 1,25-D, also known as 1,25-dihydroxycholecalciferol, 1,25-dihydroxyvitamin D₃ and calcitriol. In particularly preferred embodiments the vitamin D compound is vitamin D₃, depicted in Table II below, but the invention is not so limited. In particular embodiments the composition is provided in a dose form comprising vitamin D₃ provided in a range selected from: 500 to 4,500 I.U.; 750 to 4,250 I.U.; 1,000 to 4,000 I.U.; 1,250 to 3,750 I.U.; 1,500 to 3,500 I.U.; 1,750 to 3,250 I.U.; 2,000 to 3,000 I.U.; 2,250 to 2,750 I.U.; or about 2,500 I.U.

Preferred cobalamin compounds for the invention include: methylcobalamin;

hydroxocobalamin; cyanocobalamin; and adenosylcobalamin. Among other properties cobalamin compounds can bind the radical NO•. Methylcobalamin is particularly preferred, and is depicted in Table II below, but the invention is not so limited. In some embodiments the composition is provided in a dose form comprising a cobalamin compound provided in a range selected from: 20 to 220 μg; 30 to 210 μg; 40 to 200 μg; 50 to 190 μg; 60 to 180 μg; 70 to 170 μg; 80 to 160 μg; 90 to 150 μg; 100 to 140 μg; 110 to 130 μg; or about 120 μg.

Preferred carotenoids for purposes of the invention include the lettered carotenes, lycopene, neurosporene, phytofluene, phytoene, xanthophyll, canthaxanthin, cryptoxanthin, zeaxanthin, astaxanthin, lutein, and rubixanthin. A particularly preferred carotenoid is lutein, depicted in Table II below, but the invention is not so limited. In certain embodiments the composition is provided in a dose form comprising lutein in a range selected from: 1 to 9 mg; 1.5 to 8.5 mg; 2 to 8 mg; 2.5 to 7.5 mg; 3 to 7 mg; 3.5 to 6.5 mg; 4 to 6 mg; 4.5 to 5.5 mg; or about 5 mg.

In certain embodiments the composition is provided in a dose form that comprises per each milligram of carotenoid compound: 500 I.U. vitamin D compound and 24 μg cobalamin compound.

TABLE II
ANTIOXIDANTS WITH CONJUGATED SEGMENTS
Vitamin D3

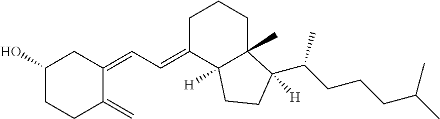 [SMILES: C=C1CC[C@H](O)C/C1=C/C=C1\CCC[C@]2(C)[C@@H]([C@H](C)CCCC(C)C)CC[C@@H]12]

Methylcobalamin

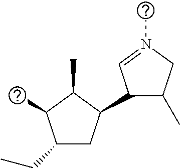

Lutein

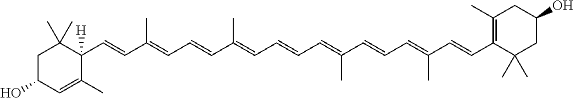 [SMILES: CC1=C[C@H](O)CC(C)(C)[C@H]1/C=C/C(C)=C/C=C/C(C)=C/C=C/C=C(C)/C=C/C=C(C)/C=C/C1=C(C)C[C@@H](O)CC1(C)C]

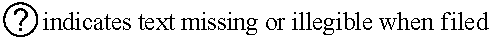

### CATEGORY III

### Compounds That Have a Disulfide Bond

Aggressive radicals can attack disulfide bonds, such as by abstracting an electron from one of the sulfurs, or by forming a bond with one of the S atoms while breaking the S—S bond and leaving the other S atom as a radical —S• that is stabilized by the electron-rich environment of the sulfur atom itself.

Particularly useful disulfides include: natural products and especially botanical natural products; derivatives of B-vitamins; and lipoic acid and its derivatives. Certain preferred disulfide antioxidants are natural products, including: lipoic acid, glutathione disulfide, and L-cystine, each of which is common in mammalian metabolism; asparagusic acid, commonly found in asparagus plants; lenthionine, commonly found in Shiitake mushrooms; and disulfide compounds from the Allium (onion and garlic) family, including ajoene, allithiamine, allyl propyl disulfide, and diallyl disulfide. Certain other preferred disulfide antioxidants are derivatives of small-molecule B vitamins, including fursultiamine (for B₁), pantethine and its salts (for B₅). prosultiamine (for B₁), pyritinol (for B₆), and sulbutiamine (for B₁), each of which is depicted in Table III below. Particularly preferred disulfide antioxidants for purposes of the invention are lipoic acid or its derivative lipoamide, but the invention is not so limited.

In certain embodiments the composition is provided in a dose form comprising alpha-lipoic acid provided in a range selected from: 100 to 900 μg; 150 to 850 μg; 200 to 800 μg; 250 to 750 μg; 300 to 700 μg; 350 to 650 μg; 400 to 600 μg; 450 to 550 μg; or about 500 μg.

TABLE III
DISULFIDES
Lipoic Acid

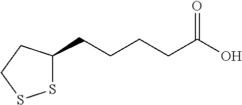 [SMILES: O=C(O)CCCC[C@@H]1CCSS1]

Lipoamide

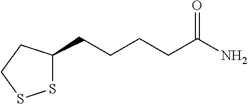 [SMILES: NC(=O)CCCC[C@@H]1CCSS1]

Asparagusic acid

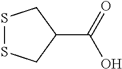

Diallyl disulfide

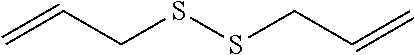

Allyl propyl sulfide

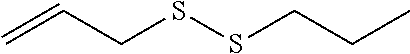

Ajoene

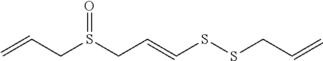

Lenthionine

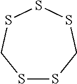

Pyritinol

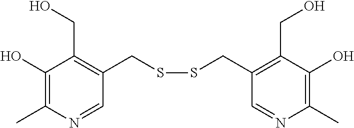 [SMILES: Cc1ncc(CSSCc2cnc(C)c(O)c2CO)c(CO)c1O]

Cystine

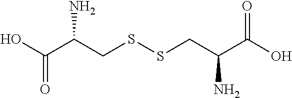 [SMILES: N[C@H](CSSC[C@H](N)C(=O)O)C(=O)O]

Allithiamine

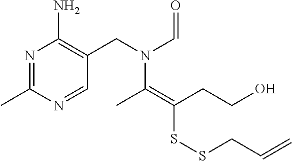 [SMILES: C=CCSS/C(CCO)=C(\C)N(C=O)Cc1cnc(C)nc1N]

Fursultiamine

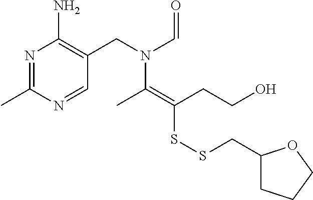 [SMILES: C/C(=C(/CCO)SSCC1CCCO1)N(C=O)Cc1cnc(C)nc1N]

Prosultiamine

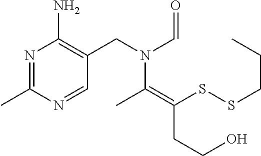 [SMILES: CCCSS/C(CCO)=C(/C)N(C=O)Cc1cnc(C)nc1N]

Sulbutiamine

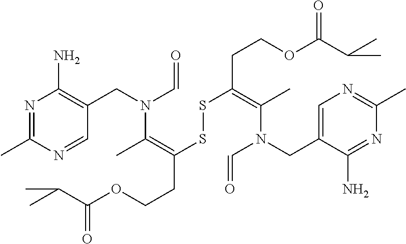

Pantethine

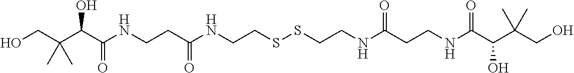 [SMILES: CC(C)(CO)[C@H](O)C(=O)NCCC(=O)NCCSSCCNC(=O)CCNC(=O)[C@H](O)C(C)(C)CO]

Glutathione disulfide

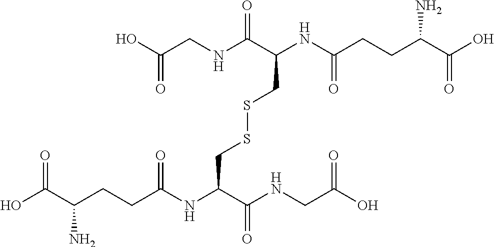 [SMILES: N[C@@H](CCC(=O)N[C@@H](CSSC[C@H](NC(=O)CC[C@H](N)C(=O)O)C(=O)NCC(=O)O)C(=O)NCC(=O)O)C(=O)O]

### CATEGORY IV

### Flavan-3-ol Compounds

Flavanol-3-ol compounds are flavonoids with numerous sites for abstraction of a phenolic hydrogen, to leave an aromatically delocalized and thus stabilized oxyradical —O•.

Preferred flavan-3-ols for purposes of the invention are depicted in Table IV below: catechin (C), epicatechin (E), gallate (G), epigallocatechin (EGC), epigallocatechin gallate (EGCG), theaflavins, theaflavin-3-gallate, and thearubigins. Molecular variants based on stereoisomers, glycosides, oligomers and polymers are also preferred, in particular proanthocyanidins and their subset the procyanidins. Particularly preferred flavan-3-ols are (+)-catechin, (+)-gallocatechin, (−)-epicatechin, (−)-epigallocatechin, (−)-epigallocatechin gallate (EGCG), (−)-epicatechin 3-gallate, and procyanidins and other proanthocyanidins.

In certain preferred embodiments the flavan-3-ols are provided in the form of a mixture found in nature, such as in grape seed extract. Grape seed extract comprises phenolic antioxidants, such as for instance, epicatechin and oligomers of epicatechin. However the invention is not so limited. Other desirable sources of mixed flavan-3ols include, for example, apples (especially Red Delicious and Granny Smith varieties), maritime pine bark, cinnamon, aronia fruit, cocoa beans, grape skins, red wine, bilberry, cranberry, black currant, green tea, black tea, oak heartwood (Quercus petraea and Q. robur), acai oil, field beans (Vicia faba), and Gallipoli rose. In various embodiments the composition is provided in a dose form that comprises grape seed extract in a range selected from: 10-90 mg; 15 to 85 mg; 20 to 80 mg; 25 to 75 mg; 30 to 70 mg; 35 to 65 mg; 40 to 60 mg; 45 to 55 mg; or about 50 mg.

TABLE IV
ILLUSTRATIVE FLAVAN-3-OLS
(+)-Catechin, C,
[Oligomers = Procyanidins]

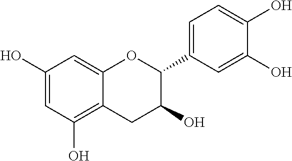 [SMILES: Oc1cc(O)c2c(c1)O[C@H](c1ccc(O)c(O)c1)[C@@H](O)C2]

(−)-Epicatechin (cis), EC
[Oligomers = Procyanidins]

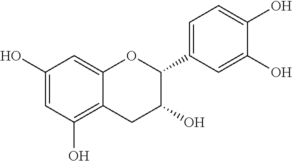 [SMILES: Oc1cc(O)c2c(c1)O[C@H](c1ccc(O)c(O)c1)[C@H](O)C2]

(−)-Epigallocatechin, EGC
[Oligomers = Prodelphinidins]

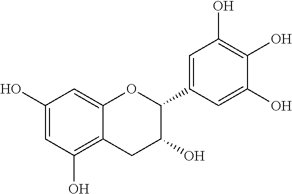 [SMILES: Oc1cc(O)c2c(c1)O[C@H](c1cc(O)c(O)c(O)c1)[C@H](O)C2]

(−)-Epicatechin gallate, ECG

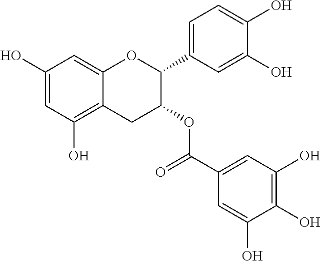 [SMILES: O=C(O[C@@H]1Cc2c(O)cc(O)cc2O[C@@H]1c1ccc(O)c(O)c1)c1cc(O)c(O)c(O)c1]

(−)-Epigallocatechin gallate, EGCG,

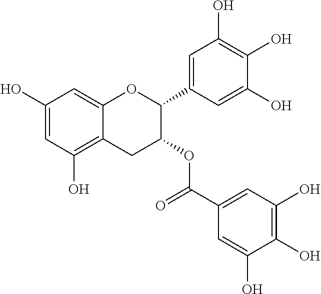 [SMILES: O=C(O[C@@H]1Cc2c(O)cc(O)cc2O[C@@H]1c1cc(O)c(O)c(O)c1)c1cc(O)c(O)c(O)c1]

Epiafzelechin

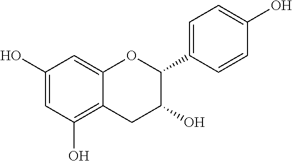 [SMILES: Oc1ccc([C@H]2Oc3cc(O)cc(O)c3C[C@H]2O)cc1]

Fisetinidol

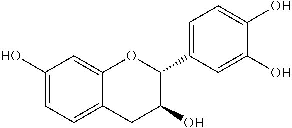 [SMILES: Oc1ccc2c(c1)O[C@H](c1ccc(O)c(O)c1)[C@@H](O)C2]

Mesquitol

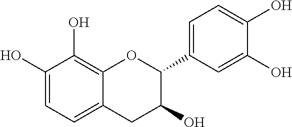 [SMILES: Oc1ccc([C@H]2Oc3c(ccc(O)c3O)C[C@@H]2O)cc1O]

Guibourtinidol
[Oligomers = Proguibourtinidins]

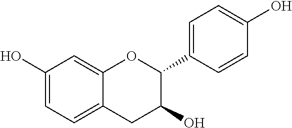 [SMILES: Oc1ccc([C@H]2Oc3cc(O)ccc3C[C@@H]2O)cc1]

Robinetinidol
[Oligomers = Prorobinetinidins]

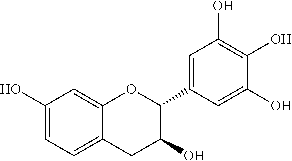 [SMILES: Oc1ccc2c(c1)O[C@H](c1cc(O)c(O)c(O)c1)[C@@H](O)C2]

### Antioxidants That are Also Pro-Oxidants

Without being bound by theory it is believed that the pro-oxidant properties of some antioxidants employed in the present invention contribute to establishing an oxidative balance in the tissues, and to maintaining an equilibrium in the oxidation states of the metals that are active there. It is also conceivable that this may help to expose and flush out latent populations of deleteriously oxidizing species found there. Among other observations, antioxidants that exert a pro-oxidant effect in the liver are particularly useful. This may possibly arise from a readier flushing of some metals from that tissue when they are in an alternative oxidation state. Comments follow on the pro-oxidant capabilities of the antioxidant classes for which use is taught here. Note that some members of an antioxidant class may be pro-oxidants while others may not be.

Most compounds with a stabilizing heteroatom do not appear to be pro-oxidants. Their role in up- and down-regulation of biochemical pathways in some cases can alter the oxidative stress. However for purposes of the invention that alone does not make them pro-oxidants.

The pro-oxidant properties of vitamin D in native tissues are not clear, though it has pro-oxidative effects in breast cancer cells. Cobalamin compounds do not appear to be pro-oxidants, nor do most carotenoids.

Antioxidants with disulfide bonds may be pro-oxidants, for instance alpha-lipoic acid is.

Flavan-3-ols behave somewhat analogously to ascorbate and commonly have prooxidant activity even at low concentrations (unlike the sterically protected phenolics such as the synthetic antioxidants BHA and BHT); this is evident for instance by their initiation of lipid peroxidation in the presence of O₂ and a metal such as Cu, Al, Zn, Ca, Mg or Cd.

### RELATIVE RATIOS IN THE COMPOSTIONS

In certain embodiments the composition is provided in a dose form in which the total weight of compounds from each of Categories, I, II, III and IV is provided in a respective ratio of 1,000:10:100:1, or wherein each number in the ratio may be varied by ±80% independently of the others.

The embodiments of the invention as described herein are merely illustrative and are not exclusive. Numerous additions, variations, derivations, permutations, equivalents, combinations and modifications of the above-described invention will be apparent to persons of ordinary skill in the relevant arts and are within the scope and spirit of the invention. The invention as described herein contemplates the use of those alternative embodiments without limitation.

1) An improved composition for treatment of prediabetes comprising therapeutically effective amounts of at least four types of antioxidant compounds, characterized in that:
a) a plurality of heteroatom-containing natural antioxidant compounds have the formula X—CH(R¹)—CH(R²)—(CH₂)ₘ—Z, wherein:
i) X is one of the following moieties or a pharmaceutically acceptable salt thereof: —NH₂ or —N(CH₃)₃;
ii) le is —H, —CO₂H, or a pharmaceutically acceptable salt of —CO₂H;
iii) R² is —H, —OH, or —O—acetyl;
iv) m is 0, 1, 2, or 3; and
v) Z is one of the following moieties or a pharmaceutically acceptable salt thereof: —SH, —SO₂H, —SO₃H, —CO₂H, —N═C(—NH₂)—NH₂, —CH(—NH₂)—CO₂H; —imidazolyl; or —C(═O)—NH—CH(—CO₂H)—CH₂—Y where Y is 4-imidazolyl or 1-methyl-4-imidazolyl;
b) a plurality of antioxidant compounds have a conjugated segment, comprising at least one vitamin D compound and at least one cobalamin compound;
c) at least one of the antioxidant compounds comprises a disulfide bond; and
d) at least one of the antioxidant compounds is a flavan-3-ol compound;
wherein at least one antioxidant compound in the composition is also a pro-oxidant.

2) The composition of claim 1 wherein the compounds having the formula X—CH₂—CH(R)—(CH₂)ₘ—Z comprise a plurality selected from the group consisting of the following compounds and their pharmaceutically acceptable salts: taurine, beta-alanine, acetyl-L-carnitine, agmatine, carnosine, cysteine, sulfinoalanine, hypotaurine, carnitine, lysine, anserine, histidine, arginine, and ornithine.

3) The composition of claim 1 wherein the compounds having the formula X—CH₂—CH(R)—(CH₂)ₘ—Z comprise at least each of taurine, beta-alanine, acetyl-L-carnitine, agmatine, and L-carnosine.

4) The composition of claim 1 wherein the plurality of antioxidant compounds having a conjugated segment comprises at least one of vitamin D₃ and methylcobalamin.

5) The composition of claim 1 wherein the plurality of antioxidant compounds having a conjugated segment further comprise a carotenoid selected from the group consisting of lettered carotenes, lycopene, neurosporene, phytofluene, phytoene, canthaxanthin, cryptoxanthin, zeaxanthin, astaxanthin, lutein, and rubixanthin.

6) The composition of claim 1 wherein the at least one antioxidant compound comprising a disulfide bond is selected from the group consisting of alpha-lipoic acid, lipoamide, glutathione disulfide, L-cystine, asparagusic acid, lenthionine, ajoene, allithiamine, allyl propyl disulfide, diallyl disulfide, fursultiamine, pantethine and its salts prosultiamine, pyritinol, and sulbutiamine.

7) The composition of claim 1 wherein the at least one antioxidant compound comprising a disulfide bond comprises alpha-lipoic acid.

8) The composition of claim 1 wherein the at least one flavan-3-ol compound is selected from the group consisting of monomers, oligomers, polymers, glycosides and pharmaceutically acceptable salts of the following: (+)-catechin, (−)-epicatechin, epigallocatechin, epicatechin gallate, epigallocatechin gallate, epiafzelechin, and fisetinidol.

9) The composition of claim 1 wherein the at least one flavan-3-ol compound is provided in the form of grape seed extract.

10) The composition of claim 1 wherein at least one of a disulfide compound or a flavan-3-ol is both an antioxidant and a pro-oxidant.

11) The composition of claim 1 wherein:
a) compounds having the formula X—CH₂—CH(R)—(CH₂)ₘ—Z comprise at least:
i) taurine in an amount selected from the range of 40 to 360 mg;
ii) beta-alanine in an amount selected from the range of 10 to 90 mg;
iii) acetyl-L-carnosine in an amount selected from the range of 20 to 180 mg;
iv) agmatine in an amount selected from the range of 25 to 225 mg; and
v) L-carnosine in an amount selected from the range of 5 to 45 mg;
b) compounds having a conjugated segment comprise at least:
i) vitamin D₃ in an amount selected from the range of 500 to 4,500 I.U.; and
ii) methylcobalamin in an amount selected from the range of 20 to 220 μg; and
c) a plurality of flavan-3-ols is provided in the form of grape seed extract in an amount selected from the range of 10 to 90 mg.

12) The composition of claim 11 wherein the composition further comprises:
a) a carotenoid, lutein, in an amount selected from the range of 1 to 9 mg; and
b) a disulfide compound, alpha-lipoic acid, in an amount selected from the range of 100 to 900 μg.

13) The composition of claim 1 wherein the composition comprises:
a) taurine in an amount of 200 mg;
b) beta-alanine in an amount of 50 mg;
c) acetyl-L-carnosine in an amount of 100 mg;
d) agmatine in an amount selected of 125 mg;
e) L-carnosine in an amount of 25 mg;
f) vitamin D₃ in an amount of 2,500 I.U.;
g) methylcobalamin in an amount of 120 μg;
h) lutein in an amount of 5 mg;
i) grape seed extract in an amount of 50 mg; and
j) alpha-lipoic acid in an amount of 500 μg.

14) A method for treatment of prediabetes comprising administration of a composition that comprises therapeutically effective amounts of at least four types of antioxidant compounds, characterized in that:
a) a plurality of heteroatom-containing natural antioxidant compounds have the formula X—CH(R¹)—CH(R²)—(CH₂)ₘ—Z, wherein:
i) X is one of the following moieties or a pharmaceutically acceptable salt thereof: —NH₂ or —N(CH₃)₃;
ii) R¹ is —H, —CO₂H, or a pharmaceutically acceptable salt of —CO₂H;
iii) R² is —H, —OH, or —O—acetyl;
iv) m is 0, 1, 2, or 3; and
v) Z is one of the following moieties or a pharmaceutically acceptable salt thereof: —SH, —SO₂H, —SO₃H, —CO₂H, —N═C(—NH₂)—NH₂, —CH(—NH₂)—CO₂H; —imidazolyl; or —C(═O)—NH—CH(—CO₂H)—CH₂—Y where Y is 4-imidazolyl or 1-methyl-4-imidazolyl;
b) a plurality of antioxidant compounds have a conjugated segment, comprising at least one vitamin D compound and at least one cobalamin compound;
c) at least one of the antioxidant compounds comprises a disulfide bond; and
d) at least one of the antioxidant compounds is a flavan-3-ol compound;
wherein at least one antioxidant compound in the composition is also a pro-oxidant.

15) The method of claim 14 wherein the compounds having the formula X—CH₂—CH(R)—(CH₂)ₘ—Z comprise a plurality selected from the group consisting of the following compounds and their pharmaceutically acceptable salts: taurine, beta-alanine, acetyl-L-carnitine, agmatine, L-carnosine, cysteine, sulfinoalanine, hypotaurine, L-carnitine, L-lysine, anserine, histidine, arginine, and ornithine.

16) The method of claim 14 wherein the compounds having the formula X—CH₂—CH(R) —(CH₂)ₘ—Z comprise at least each of taurine, beta-alanine, acetyl-L-carnitine, agmatine, and L-carnosine.

17) The method of claim 14 wherein the plurality of antioxidant compounds having a conjugated segment comprises at least one of vitamin D₃ and methylcobalamin.

18) The method of claim 14 wherein the plurality of antioxidant compounds having a conjugated segment further comprise a carotenoid selected from the group consisting of carotenes, lycopene, neurosporene, phytofluene, phytoene, canthaxanthin, cryptoxanthin, zeaxanthin, astaxanthin, lutein, and rubixanthin.

19) The method of claim 14 wherein the at least one antioxidant compound comprising a disulfide bond is selected from the group consisting of alpha-lipoic acid, lipoamide, glutathione disulfide, L-cystine, asparagusic acid, lenthionine, ajoene, allithiamine, allyl propyl disulfide, diallyl disulfide, fursultiamine, pantethine and its salts prosultiamine, pyritinol, and sulbutiamine.

20) The method of claim 14 wherein the at least one antioxidant compound comprising a disulfide bond comprises alpha-lipoic acid.

21) The method of claim 14 wherein the at least one flavan-3-ol compound is selected from the group consisting of monomers, oligomers, polymers, glycosides and pharmaceutically acceptable salts of the following: (+)-catechin, (−)-epicatechin, epigallocatechin, epicatechin gallate, epigallocatechin gallate, epiafzelechin, and fisetinidol.

22) The method of claim 14 wherein the at least one flavan-3-ol compound is provided in the form of grape seed extract.

23) The method of claim 14 wherein at least one of a disulfide compound or a flavan-3-ol is both an antioxidant and a pro-oxidant.

24) The method of claim 14 wherein:
a) compounds having the formula X—CH₂—CH(R)—(CH₂)ₘ—Z comprise at least:
i) taurine in an amount selected from the range of 40 to 360 mg;
ii) beta-alanine in an amount selected from the range of 10 to 90 mg;
iii) acetyl-L-carnosine in an amount selected from the range of 20 to 180 mg;
iv) agmatine in an amount selected from the range of 25 to 225 mg; and
v) L-carnosine in an amount selected from the range of 5 to 45 mg;
b) compounds having a conjugated segment comprise at least:
i) vitamin D₃ in an amount selected from the range of 500 to 4,500 I.U.; and
ii) methylcobalamin in an amount selected from the range of 20 to 220 μg; and
c) a plurality of flavan-3-ols is provided in the form of grape seed extract in an amount selected from the range of 10 to 90 mg.

25) The method of claim 24 wherein the composition further comprises:
a) a carotenoid, lutein, in an amount selected from the range of 1 to 9 mg; and
b) a disulfide compound, alpha-lipoic acid, in an amount selected from the range of 100 to 900 μg.

26) The method of claim 14 wherein the composition comprises:
a) taurine in an amount of 200 mg;
b) beta-alanine in an amount of 50 mg;
c) acetyl-L-carnosine in an amount of 100 mg;
d) agmatine in an amount selected of 125 mg;
e) L-carnosine in an amount of 25 mg;
f) vitamin D₃ in an amount of 2,500 I.U.;
g) methylcobalamin in an amount of 120 μg;
h) lutein in an amount of 5 mg;
i) grape seed extract in an amount of 50 mg; and
j) alpha-lipoic acid in an amount of 500 μg.
